(19) United States
(12) Patent Application Publication
(10) Pub. No.: US 2017/0048651 A1
(43) Pub. Date: Feb. 16, 2017

(54) STATE DETERMINATION APPARATUS AND SYSTEM FOR A CARE-RECEIVER

(71) Applicant: Hello Nurse Medical Innovation, Inc. Taiwan Branch, New Taipei City (TW)

(72) Inventors: Sharon Lin Charna, New Taipei City (TW); Chia Hsi Chen, New Taipei City (TW)

(73) Assignee: Hello Nurse Medical Innovation, Inc. Taiwan Branch, New Taipei City (TW)

(21) Appl. No.: 15/233,985

(22) Filed: Aug. 11, 2016

(30) Foreign Application Priority Data
Aug. 13, 2015 (TW) 104213035

(51) Int. Cl. H04W 4/00; A61B 5/00; A61F 13/42; H04B 1/3888; A61B 5/11

(52) U.S. Cl. CPC H04W 4/008; H04B 1/3888; A61B 5/1117; A61F 13/42; A61B 5/0022; H04B 2001/3894

(57) ABSTRACT

A state determination apparatus for a care-receiver is provided, comprising: a communication unit and a protective unit. The communication unit includes at least one first antenna module and a first wireless communication module. The protective unit is configured for accommodating the communication unit and buffering external pressure on the communication unit to protect the communication unit. The state determination apparatus is configured for being disposed on a back of a cushion. The first wireless communication module sends a detection signal through at least the first antenna module so as to communicate with a radio frequency identity (RFID) tag used by the care-receiver. After sending the detection signal, the communication unit determines whether a response signal from the RFID tag is received or information of the response signal is read so as to determine a state of the care-receiver.

### CROSS-REFERENCES TO RELATED APPLICATION

This non-provisional application claims priority under 35 U.S.C. §119(a) from Patent Application No. 104213035 filed in Taiwan, R.O.C. on Aug. 13, 2015, the entire contents of which are hereby incorporated by reference.

### BACKGROUND

Technical Field

The invention relates to technology of determining a state of a care-receiver, and in particular, to state determination apparatus and system for a care-receiver to detect such as a wetness state of an absorbent article for a care-receiver, a fall warning state for a care-receiver, wherein the absorbent article may be any diaper (such as adults diapers), urine pads.

Related Art

The technology of diapers equipped with detection devices for wetness detection arises from the needs for long-term care for babies, patients, or disabled elders so as to reduce the burden of work on the caregivers and to deliver better services to the care-receivers.

Conventional diapers with detection devices are complicated in structure. Some of them require sensors and antenna disposed on one side of the diaper in contact with users' skin, which may make the users feel uncomfortable. In addition to requiring the users using such diapers, a determination device, which may have the similar size as a telephone, is required to be disposed on the bed or a fixed position on the room in order to inform the caregivers of whether the diaper is wet. Such systems of detection and determination occupy space and require high hardware cost.

### SUMMARY

Accordingly, state determination apparatus and system for a care-receiver are provided, which can be employed to determine a state of a care-receiver, such as a wetness state of an absorbent article and/or a state of fall warning for the care-receiver, and so on. The absorbent article may be any type of absorbent articles, for example, diapers (such as adults' diaper, babies' diapers, and so on), urine pads, and so on.

According to an embodiment of the invention, provided is a state determination apparatus for a care-receiver, comprising: a communication unit and a protective unit. The communication unit includes a first antenna module and a first wireless communication module, the first wireless communication module and the first antenna module are electrically coupled. The protective unit, which at least covers the communication unit, is configured for accommodating the communication unit and for buffering external pressure on the communication unit so as to protect the communication unit. The state determination apparatus for a care-receiver is configured for being disposed on a back of a cushion. The first wireless communication module sends a detection signal through at least the first antenna module so as to communicate with a radio frequency identity (RFID) tag. After the detection signal is sent, the communication unit determines whether a response signal is received or information of the response signal is read so as to determine a state of the care-receiver.

In an embodiment, the communication unit of the state determination apparatus for a care-receiver further comprises:a second antenna module and a second wireless communication module;the second wireless communication module is electrically coupled to the first wireless communication module and the second antenna module; wherein the first wireless communication module utilizes the second antenna module and the second wireless communication module so as to communicate with a computing device in a wireless network, and sends the state of the care-receiver to the computing device.

According to another embodiment of the invention, a state determination system for a care-receiver is provided, comprising:an RFID tag and the state determination apparatus for a care-receiver according to any of the above embodiments. The state determination apparatus for a care-receiver is utilized for being disposed on a back of a cushion.

In an embodiment, the RFID tag is disposed on an outer side of an absorbent article, the state of the care-receiver is, such as a wetness state of the absorbent article of the care-receiver. The absorbent article may be a diaper or urine pad.

In an embodiment, the RFID tag is worn on the care-receiver or attached to clothes of the care-receiver, and the state of the care-receiver is a fall warning state of the care-receiver.

For better understanding of the above and other aspects of the invention, a plurality of embodiments or examples will be taken with accompanying drawings to provide detailed description as follows.

### BRIEF DESCRIPTION OF THE DRAWINGS

FIGS. 1A-1C illustrate schematic diagrams of a state determination apparatus for a care-receiver according to an embodiment of the invention.

FIGS. 2A-2B illustrate schematic diagrams of an embodiment of the state determination apparatus for a care-receiver, disposed on a mattress.

FIG. 3 illustrates an RFID tag disposed on a diaper.

FIGS. 4A-4B illustrate schematic diagrams of an embodiment of the state determination apparatus for a care-receiver, disposed on different kinds of cushions.

FIG. 5 is a schematic diagram illustrating the state determination apparatus for a care-receiver and a computing device of a healthcare center, according to an embodiment.

FIGS. 6A-6D illustrate cross-sectional views of embodiments of the state determination apparatus for a care-receiver.

FIG. 7 illustrates a schematic diagram of an embodiment of an input part of the state determination apparatus for a care-receiver.

FIG. 8 illustrates a schematic diagram of an embodiment of the state determination apparatus for a care-receiver.

FIG. 9 is a schematic diagram illustrating an embodiment of using the state determination apparatus for a care-receiver with an RFID tag disposed on a pair of trousers.

FIG. 10 illustrates a schematic diagram of another embodiment of a state determination apparatus for a care-receiver with multiple antennas for communication with an RFID tag.

FIG. 11 illustrates a schematic diagram of an embodiment of the state determination apparatus for a care-receiver, disposed on a mattress.

### DETAILED DESCRIPTION

In this detailed description of the various embodiments, for purposes of explanation, numerous specific details are set forth to provide a thorough understanding of the embodiments disclosed. One skilled in the art will appreciate, however, that these various embodiments may be implemented with or without these specific details. Furthermore, one skilled in the art can readily appreciate that it is contemplated that the embodiments can be modified or varied and still remain within the spirit and scope of the various embodiments disclosed herein.

The following will provide a plurality of embodiments so as to describe different aspects of implementations of the state determination apparatus for a care-receiver.

According to an embodiment of the invention, provided is a state determination apparatus for a care-receiver, comprising: a communication unit and a protective unit. The communication unit includes a first antenna module and a first wireless communication module; the first wireless communication module and the first antenna module are electrically coupled. The protective unit, which covers at least the communication unit, is for accommodating the communication unit and for buffering external pressure on the communication unit so as to protect the communication unit. The state determination apparatus for a care-receiver is utilized for being disposed on a back of a cushion. The first wireless communication module sends a detection signal through at least the first antenna module so as to communicate with a radio frequency identity (RFID) tag disposed on a diaper. After the detection signal is sent, the communication unit determines whether a response signal is received or information of the response signal is read so as to determine a state of the care-receiver.

The communication unit of the state determination apparatus for a care-receiver may further include: a second antenna module and a second wireless communication module. The second wireless communication module is electrically coupled to the first wireless communication module and the second antenna module; wherein the first wireless communication module utilizes the second antenna module and the second wireless communication module so as to communicate with a computing device in a wireless network, and sends the state of the care-receiver to the computing device.

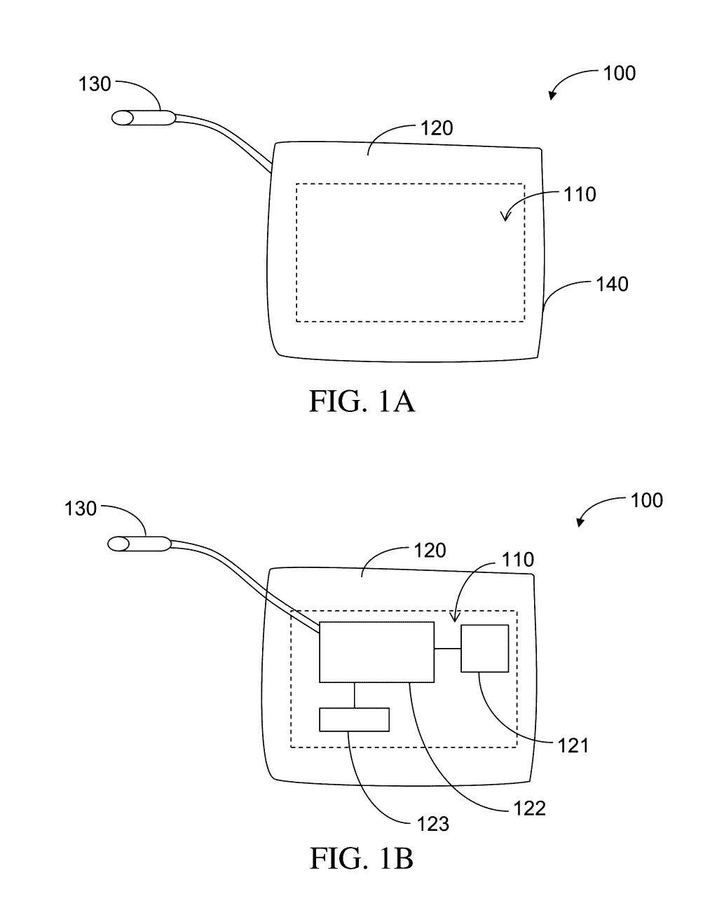

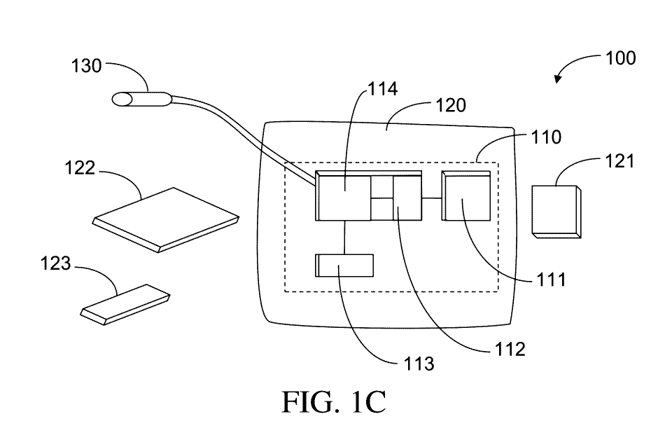

FIGS. 1A-1C illustrate schematic diagrams of a state determination apparatus for a care-receiver according to an embodiment of the invention. As shown in FIG. 1A, a state determination apparatus for a care-receiver 100 includes a communication unit 110 (wherein a region inside is indicated in dashed lines), a protective unit 120, and an input part 130. The communication unit 110 is disposed inside the protective unit 120. In addition, for the sake of waterproof and safety, the state determination apparatus for a care-receiver 100 further includes a protective cover 140. The protective cover 140 wraps the communication unit 110 and the protective unit 120. The input part 130, electrically coupled to the communication unit 110, serves as an input interface, which, for example, includes at least one of a power input, a data interface, an input key.

FIG. 1B is a schematic diagram illustrating the state determination apparatus for a care-receiver 100 when the protective cover 140 is removed. The protective unit 120 covers the communication unit 110, and a liftable part, such as liftable parts 121, 122, and 123, is provided on a region in the protective unit 120 corresponding to at least one portion of the communication unit. As shown in FIG. 1C, when any one of the liftable parts 121, 122, and 123 is lifted, the portion of the communication unit (such as the circuit or antenna) is exposed, and thus maintenance technicians or engineers can perform detection or maintenance tasks with respect to the portion of the communication unit (such as the circuit or antenna).

For example, in FIG. 1C, the communication unit 110 includes a first antenna module 111, a first wireless communication module 112, a second antenna module 113, and a second wireless communication module 114. The first wireless communication module 112 and the first antenna module 111 are electrically coupled.

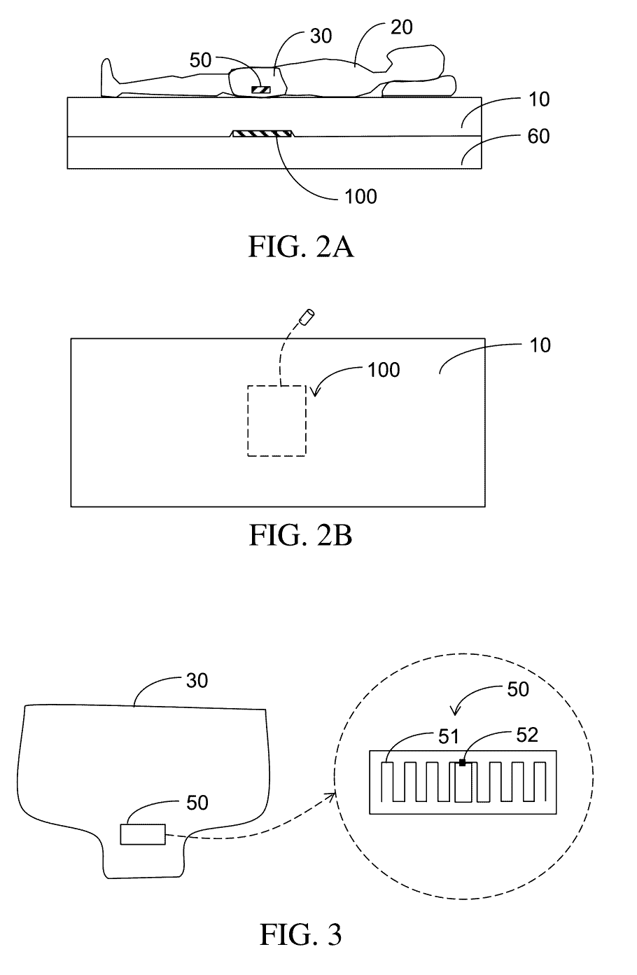

The state determination apparatus for a care-receiver according to the invention can be employed to determine the state of a care-receiver, such as a wetness state of an absorbent article and/or a state of fall warning for the care-receiver, and so on. The absorbent article may be any type of absorbent articles, for example, diapers (such as adults' diaper, babies' diapers, and so on), urine pads, and so on. For the sake of illustration, the following embodiments will take a diaper as the absorbent article. As shown in FIG. 2A, the state determination apparatus for a care-receiver 100 is utilized for being disposed on the back of a cushion 10 (such as a mattress) so as to perform state determination on a diaper 30 on a user 20 (so called a care-receiver) lying on a front surface of the cushion 10 (such as a mattress). FIG. 2B is a top view illustrating the state determination apparatus for a care-receiver 100a disposed on the back of the mattress, wherein an input part 130 can be exposed outside the mattress. FIG. 3 is a schematic diagram illustrating an RFID tag 50 disposed on the diaper 30.

Referring to FIG. 2A, the first wireless communication module 112 sends a detection signal through at least the first antenna module 111 so as to communicate with the RFID tag 50 disposed on the diaper 30. The RFID tag 50, after receiving the detection signal, will send a response signal. Accordingly, after sending the detection signal, the communication unit 110 determines whether a response signal is received or information of the response signal is read so as to determine a wetness state of the diaper 30. In an embodiment, the communication unit 110 utilizes the first wireless communication module 112 (or the second wireless communication module 114) for determining whether a response signal indicating the diaper in a wetness state is received. For example, if the response signal is received, it is indicated that the diaper 30 is in a wetness state of getting wet; if the response signal is not received, it is indicated that the diaper 30 is not wet. In another embodiment, the communication unit 110 reads the information of the response signal by the first wireless communication module 112 (or the second wireless communication module 114); the information of the response signal can be configured to include a wetness state value, for example, the value ranging from 0 to 100 to indicate the states from dry to fully wet, respectively; and the wetness state of the diaper 30 can then be obtained by reading the wetness state value in the response signal. In addition, in another embodiment, if the communication unit 110, after sending a detection signal for one or more times, does not receive any response signal (or receiving the response signal which is less than a normal value or less than a lower limit of a normal range), it can further determine that the fall warning state of the user 20 with the diaper 30 is “abnormal” (which is another type of state of the care-receiver), which may indicate that the user 20 probably has left the position of the cushion (e.g., a mattress, a wheelchair cushion, or a seat cushion), or the user 20 has fallen or something wrong has happened to the user 20. Hence, the second antenna module 113 and the second wireless communication module 114 can be utilized for further sending a warning signal to call caregivers to observe what was happening on the spot, for the sake of safety.

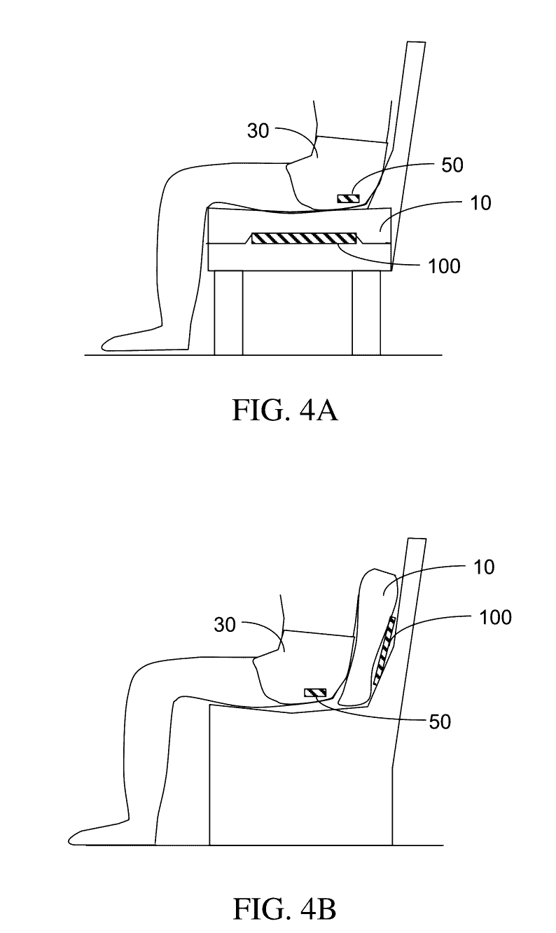

In addition to being disposed on the back of a mattress as exemplified above, the state determination apparatus for a care-receiver can be disposed on a back portion of any cushion (or the like), such as a wheelchair cushion, a back support cushion, a seat cushion, a lumbar cushion, and an air cushion. As shown in FIG. 4A, the state determination apparatus for a care-receiver 100 is disposed under a cushion 10, such as a wheelchair cushion, a back support cushion, a seat cushion, and an air cushion, in some embodiments. As shown in FIG. 4B, the state determination apparatus for a care-receiver 100 is disposed on a cushion 10, such as a lumbar cushion.

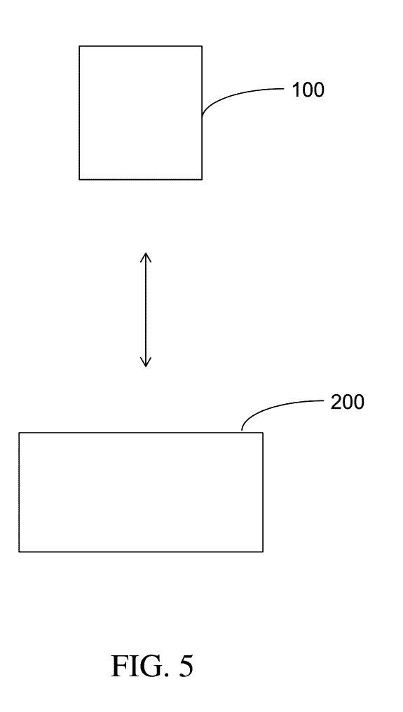

Referring to FIG. 1C, the second wireless communication module 114 of the state determination apparatus for a care-receiver 100 is electrically coupled to the first wireless communication module 112 and the second antenna module 113. The first wireless communication module 112 communicates with a computing device in a wireless network by the second antenna module 113 and the second wireless communication module 114, and sends the state of the care-receiver to the computing device. The computing device refers to any electronic device with communication, computing power, such as cloud servers, LAN servers, desktop computers, laptop computers, tablet computers, and even smartphones, PDAs, pagers, dedicated communications equipment and so on. For example, FIG. 5 illustrates a state determination apparatus for a care-receiver according to an embodiment communicating with a computer of a healthcare center. In an embodiment, an additional piece of wireless network equipment, such as a wireless router, can be utilized as a relay (or bridging) device between the state determination apparatus for a care-receiver and the computing device. The second antenna module 113 and the second wireless communication module 114 can be implemented to be compliant with one or more wireless network standards, such as one or any combination of Zigbee, Wi-Fi, Bluetooth, mobile communication protocols (e.g., GSM, CDMA, WCDMA, UMTS, LTE and so on), satellite navigation system (e.g., GPS of the US, Galileo of EU, Beidou of People's Republic of China).

In addition to execute an application program to display the state of the care-receiver so as to remind the caregiver (nursing staff) for further processing, the computing device can be further configured by an application program or program modules to record data, such as data about urination or defecation of the care-receiver (such as a bedridden person), which can be further tracked for indication of the care-receiver's condition and/or other application. In addition to the computing device, as exemplified above, a server or cloud data center can be utilized for the implementation of, for example, a software system communicating care-receivers and caregivers (e.g., HelloNurse™ system) so as to realize the functionality of recording data of caring or warning. Moreover, in yet another embodiment, the care-receiver's state can be provided to an electronic device, such as smart devices, of the care-receiver's caregiver or family member, being linked to the server or cloud data center through a network. In still another embodiment, the computing device may be any smart devices, such as smartphones, tablet computers, smart watches, wearable devices, and the computing device can be configured to execute an application program (APP) for use with the state determination apparatus for a care-receiver; and in this way, the caregiver or family member can obtain the care-receiver's state or record by using the computing device through a wireless network (such as Wi-Fi) or Internet and so on. Hence, according to the embodiments of the invention, the state determination apparatus for a care-receiver can be applied to home care, in addition to healthcare centers, hospitals and so on.

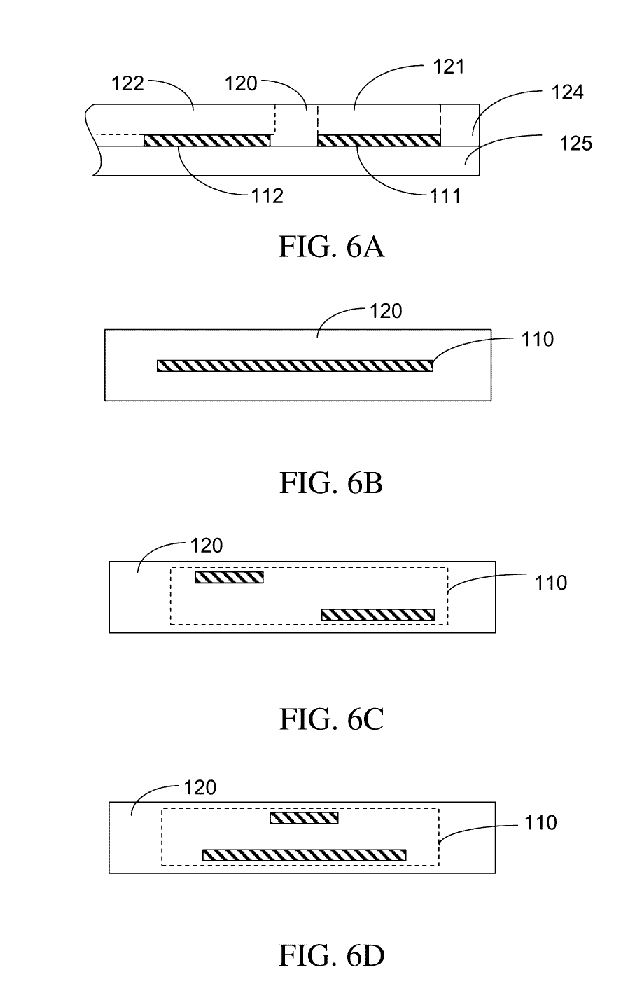

Further, as illustrated in FIGS. 1A to 1C, the protective unit 120 covers the communication unit 110, for accommodating the communication unit 110 and for buffering external pressure on the communication unit 110 so as to protect the communication unit 110. FIG. 6A is a cross-sectional view illustrating an embodiment of the state determination apparatus for a care-receiver 100 in FIG. 1B. As shown in FIG. 6A, the protective unit 120 includes a first protective layer 124 and a second protective layer 125. The communication unit 110 (e.g., the first antenna module 111, the first wireless communication module 112) is disposed between the first protective layer 124 and the second protective layer 125. The first protective layer 124 and the second protective layer 125 are flexible and can be made from flexible materials such as any of foam, rubber, paper, plastarch material, or polymer materials, or combinations thereof. In an embodiment, the first protective layer 124 has a hardness greater than that of the second protective layer 125. However, any of the embodiments is for the sake of illustration only, without imposing any limitations on the implementation of the invention; the protective unit(s) may be formed by any material, the number of the protective layer, and/or the hardness of the protective layer may be implemented in various ways in view of the design and requirements for products.

In addition, as shown in FIG. 6A, the liftable parts 121 and 122 is provided on regions, in the first protective layer 124 or second protective layer 125 of the protective unit 120, corresponding to at least one portion of the communication unit 110 (e.g., the first antenna module 111 and the first wireless communication module 112). When any of the liftable part 121 or 122 is lifted, the first antenna module 111 or the first wireless communication module 112 of the communication unit 110 can be exposed.

The arrangement of the protective unit and communication unit may be changed in other ways. FIGS. 6B to 6D show cross-sectional views of other embodiments of the state determination apparatus for a care-receiver. As shown in FIG. 6B, the communication unit 110 can be configured as a circuit board and embedded into the protective unit 120. As illustrated in FIG. 6C or 6D, the communication unit 110 can be configured as two or more modules, and the modules can be embedded on different surfaces inside the protective unit 120.

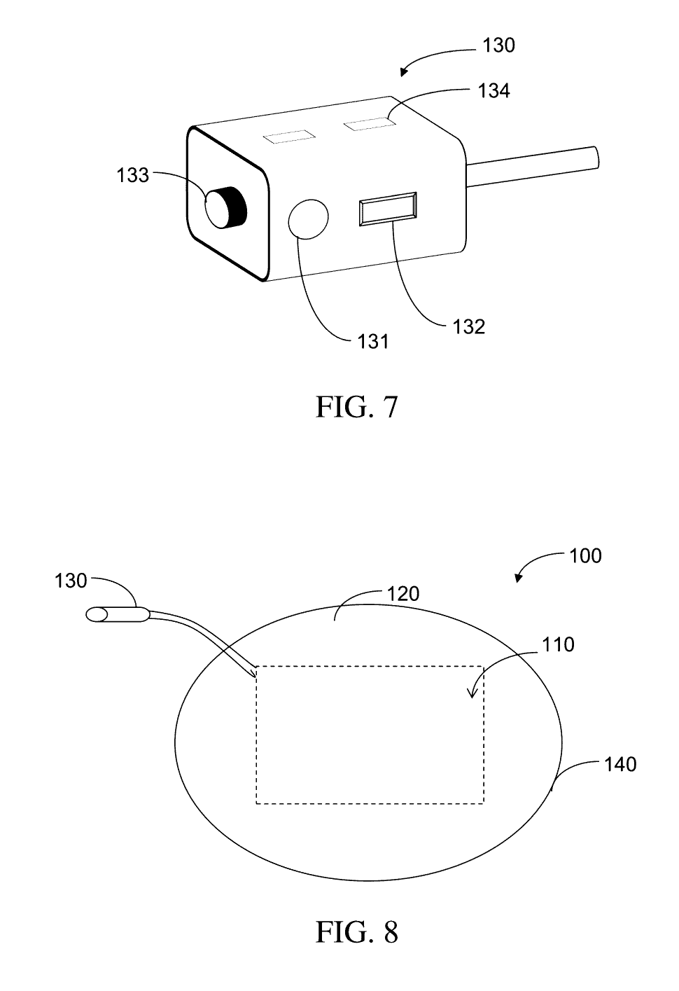

FIG. 7 illustrates a schematic diagram of an embodiment of an input part for the state determination apparatus for a care-receiver. As shown in FIG. 7, the input part 130 at least includes a power input 131, a communication interface 132 (e.g., an interface compliant to one of the data communication standards, such as RS-232, RS-422, or USB, USB2.0, USB3.0, and so on), and an input key 133 (e.g., a button key). In addition, the input part 130 may include an indicator 134 (e.g., an LED). For example, the power input 131 can be employed to an input terminal for AC or DC power for the state determination apparatus for a care-receiver; the communication interface 132 can be used for data transfer such as firmware update for the state determination apparatus for a care-receiver. In addition, the input key 133 can be used for power start-up, shutdown, and/or setting for the state determination apparatus for a care-receiver. One or more indicators 134 can be employed to indicate the operation state of the state determination apparatus for a care-receiver (e.g., normal, abnormal) or to indicate the state of the care-receiver (e.g., dryness, wetness, and so on). In addition, the above examples of the input part are optional and can be selectively implemented, without imposing any limitations on the implementation of the state determination apparatus for a care-receiver, and the apparatus can be modified or configured in other ways. For example, in another embodiment, the state determination apparatus for a care-receiver can be equipped with built-in batteries, and/or can be updated its firmware wirelessly by using the communication unit of the state determination apparatus for a care-receiver, so that the input part may be unnecessary to be included therein.

In addition, the state determination apparatus for a care-receiver according to the invention may be configured in any shapes. For example, in FIG. 8, the state determination apparatus for a care-receiver has its appearance configured to be round or oval shape.

The following further provides embodiments to illustrate how the state determination apparatus for a care-receiver 100 determines a wetness state of the diaper 30 by way of communication with the RFID tag 50 attached to the diaper 30, as shown in FIG. 3.

Referring to FIG. 3, the RFID tag 50 includes an antenna 51 and a communication chip 52. The characteristic of the antenna 51 changes according to the magnitude of the relative permittivity of the material of the substrate to which the antenna 51 is attached. That is, as the magnitude of the relative permittivity of the material of the substrate to which the antenna 51 is attached changes, the wavelength of the electromagnetic wave signal sent by the antenna 51 will change as well.

In FIG. 3, the RFID tag 50 can be attached to an outer side of the diaper 30. Specifically, a diaper generally includes a fabric layer, an absorbent material layer, and an anti-leak layer; the fabric layer is in contact with the skin of the user, and the urine of the user may pass through the fabric layer and thus is absorbed by the absorbent material layer. The outer side of diaper 30 is a side not in contact with the skin of the user; such as an outer side of the anti-leak layer of the diaper 30. In a situation that an inner side of the anti-leak layer gets the urine from the surface of the absorbent material layer, the magnitude of the relative permittivity of the anti-leak layer varies accordingly. Thus, when the inner side of the anti-leak layer has not got the urine, an electromagnetic wave signal sent by the RFID tag 50 has a wavelength very different from that of an electromagnetic wave signal sent by the RFID tag 50 when the inner side of the anti-leak layer has got the urine.

The first wireless communication module 112 sends a detection signal through at least the first antenna module 111 so as to communicate with the RFID tag 50 on the diaper 30. In an embodiment, the communication unit 110 stores a setting value, which is a numerical value of the wavelength of an electromagnetic wave (e.g., a response signal) to be transmitted by the communication unit 110 when the inner side of the anti-leak layer is not wetted. In this embodiment, when the user of the diaper 30 urinates and the diaper 30 gets wet, the impedance value of the antenna of the RFID tag 50 will change. In this case, after receiving the detection signal, the RFID tag 50 sends a response signal which has a wavelength different from that of an electromagnetic wave signal sent by the RFID tag 50 when the inner side of the anti-leak layer has not got wet. Thus, the communication unit 110, when receiving the response signal, can compare the response signal with the setting value so as to determine a wetness state of the diaper 30. In addition, in another embodiment, the setting value may set to a numerical value of the wavelength of an electromagnetic wave (e.g., a response signal) to be transmitted by the communication unit 110 when the inner side of the anti-leak layer is wetted.

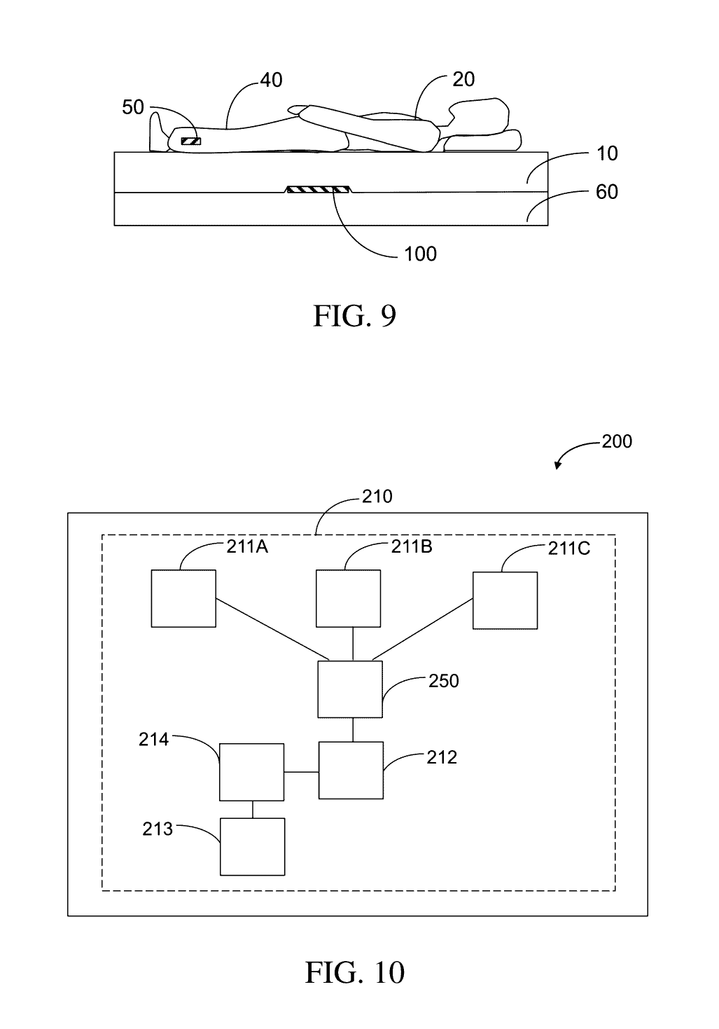

In addition, the state determination apparatus for a care-receiver according to the invention can be utilized for determining a state of a care-receiver such as a fall warning state. In some embodiments, the state determination apparatus for a care-receiver 100 can be disposed under the cushion and the RFID tag 50 can be disposed on the care-receiver; for example, the RFID tag 50 may be worn or attached to any clothes of the care-receiver (such as clothing, underwear, trousers, socks). For example, as shown in FIG. 9, an RFID tag 50 is attached to a leg of a pair of trousers 40 of the user 20 (i.e., the care-receiver). When the communication unit 110 of the state determination apparatus for a care-receiver 100, after sending a detection signal for one or a plurality of times, does not receive any response signal (or receiving the response signal which is less than a normal value or less than a lower limit of a normal range), it can further determine that the fall warning state of the user 20 is “abnormal” (which is a type of state of the care-receiver), which may indicate that the user 20 probably has left the position of the cushion (e.g., a mattress, a wheelchair cushion, or a seat cushion), or the user 20 has fallen or something wrong has happened to the user 20. Hence, the second antenna module 113 and the second wireless communication module 114 can be utilized for further sending a warning signal to call caregivers to observe what was happening on the spot, for the sake of safety. Conversely, if the communication unit 110 does receive a response signal (or receiving the response signal which is not less than a normal value or within a normal range), it can be determined that the fall warning state of the user 20 is “normal”, indicating that the user 20 remains in the position (e.g., the position of the cushion).

The following provides some embodiments of how the communication unit determines the state of the care-receiver. In implementation, the state determination can be performed in the first wireless communication module and/or the second wireless communication module of the communication unit.

In some embodiments, the communication unit determines the state of the care-receiver as an abnormal state or a state of getting wet when the response signal is not received; the communication unit determines the state of the care-receiver as a normal state or a state of getting dry when the response signal is received. In some examples, the first wireless communication module sends a detection signal through at least the first antenna module for at least one or a plurality of times so as to communicate with the RFID tag. In an example, the results of whether the response signal is received or not can be averaged in the case that the detection signal is sent for a plurality times so as to determine the state of the care-receiver. For example, a setting value (or a threshold value) can be taken as a reference that if the average occurrence of the response signal is lower than the setting value, it is determined that the state of the care-receiver is abnormal or is a state of getting wet; otherwise, it is determined that the state is normal or indicates that it is not wet.

In some embodiments, the communication unit determines the state of the care-receiver as an abnormal state or a state of getting wet when the information including an identification code in the response signal is not received; the communication unit determines the state of the care-receiver as a normal state or a state of getting dry when the information including the identification code in the response signal is received. For example, the identification code may be any code for identifying the RFID tag, such as electronic product code (EPC) or tag ID (TID) in a response signal. In some examples, the first wireless communication module sends a detection signal through at least the first antenna module for at least one or a plurality of times so as to communicate with the RFID tag. In an example, the results of whether the identification code in the response signal is received or not can be averaged in the case that the detection signal is sent for a plurality times so as to determine the state of the care-receiver. For example, a setting value (or a threshold value) can be taken as a reference that if the average occurrence of the identification code is lower than the setting value, it is determined that the state of the care-receiver is abnormal or is a state of getting wet; otherwise, it is determined that the state is normal or indicates that it is not wet.

Further, the identification code can be utilized as a tool for identification and/or calibration when the user changes the absorbent article (such as a new diaper or urine pad) or an RFID tag is changed so that the state determination apparatus identifies which RFID tag is to be detected. In a case, once a new diaper or RFID tag is used, the user can reset the state determination apparatus e.g., by pressing a button of the input part or instructing the apparatus wirelessly, so that the state determination apparatus stores the identification code of the RFID tag for identification purpose, and/or stores an initial value (or state) for the RFID tag, which can be regarded as an initial value (or state) for the care-receiver and can be used in comparison with the values (or states) for the RFID detected afterwards for state determination.

In some embodiments, the communication unit determines the state of the care-receiver based on signal strength of the response signal, such as a received signal strength indicator (RSSI) in the response signal. For example, the state of the care-receiver is a wetness state of an article on the care-receiver; and the communication unit determines the wetness state as a state of getting wet when a value based on the RSSI is within a setting range (e.g., −30 dbW to −62 dbW). The value based the RSSI indicates a corresponding amount of moisture of the article (e.g., −30 dbW for being dry initially; and −62 dbW for fully wet). In some examples, the first wireless communication module sends a detection signal through at least the first antenna module for at least one or a plurality of times so as to communicate with the RFID tag. In an example, the results of the signal strength (e.g., RSSI) can be averaged in the case that the detection signal is sent for a plurality times (e.g., 2, 3, 4, 5, 8, 10, or 30, and so on) so as to determine the state of the care-receiver or the amount of moisture of the article on the care-receiver. The following table shows some examples for the relationship between the signal strength (e.g., values in dbW) and moisture; and the invention is not limited thereto; e.g. different number of scales (10, 20, 50, or 100 or more) can be obtained or made by way of experiment or calculation.

TABLE 1

| Moisture in the | Example 1 | Example 2 | Example 3 |
|---|---|---|---|
| article (relative) | RSSI (dbW) | RSSI (dbW) | RSSI (dbW) |
| 0% (initial value) | −30 | −30 | −20 |
| 25% | −37 | −35 | −27 |
| 50% | −45 | −48 | −36 |
| 75% | −54 | −57 | −44 |
| 100% | −60 | −63 | −52 |

In other embodiments, the state determination apparatus for a care-receiver may be configured to have multiple antennas for communication with an RFID tag.

FIG. 10 illustrates a schematic diagram of another embodiment of a state determination apparatus for a care-receiver 200 with multiple antennas for communication with an RFID tag. The state determination apparatus for a care-receiver 200 may be implemented based on any of the embodiments of the above state determination apparatus or any combination thereof. As compared to the embodiment of the state determination apparatus in FIGS. 1A-1C, the main difference is that the state determination apparatus for a care-receiver 200 in FIG. 10 includes multiple antennas for communication with an RFID tag. In FIG. 10, the state determination apparatus 200 comprises a communication unit 210 having a plurality of first antenna modules (such as those indicated by 211A, 211B, and 211C), a first wireless communication module 212, a second antenna module 213, and a second wireless communication module 214. In addition, the communication unit 210 includes a switch device 250, coupled between the first antenna modules (211A, 211B, and 211C) and the first wireless communication module 212. The first wireless communication module 212 can be configured to send a detection signal through the first antenna modules for a plurality of times (e.g., 2, 3, 4, 5, 8, 10, or 30, and so on) so as to communicate with an RFID tag used by the care-receiver (e.g., the RFID tag disposed on an article such as a diaper), wherein the detection signal sent to one of the first antenna modules may be in different frequency from the detection signal sent to another one. For instance, a detection signal is repeatedly sent for a number of times to each of the first antenna modules 211A, 211B, and 211C, respectively; the detection signal may indicate a request for an identification code and/or signal strength, or other parameter(s). After the detection signal is sent, the communication unit (e.g., using the first wireless communication module 212 and/or the second wireless communication module 214) can determine a state of the care-receiver based on the response signal accordingly, for example, in the way of any of the above embodiments (e.g., presence/absent of the response signal; using signal strength; or presence/absent of an identification code, and so on, by average or any statistic approach) or any combination thereof.

In some embodiment, the first antenna modules 211A, 211B, and 211C may be operated in different operating frequencies in a frequency band; they operate in three different frequencies in a frequency band of UHF from 902 MHz to 923.75 MHz or other frequency band, but the invention is not limited thereto. The first wireless communication module 212 can be configured to send a detection signal for a plurality of times through the first antenna modules 211A, 211B, and 211C in any order, for example, in a consecutive sequence, an alternating sequence, a random sequence, or any specific sequence, or any one of them repeatedly. In an example, the first wireless communication module 212 sends a detection signal for a plurality of times through the first antenna modules 211A, 211B, and 211C with the switch device 250 for switching the operating frequency and selecting the antenna modules in an order adopted.

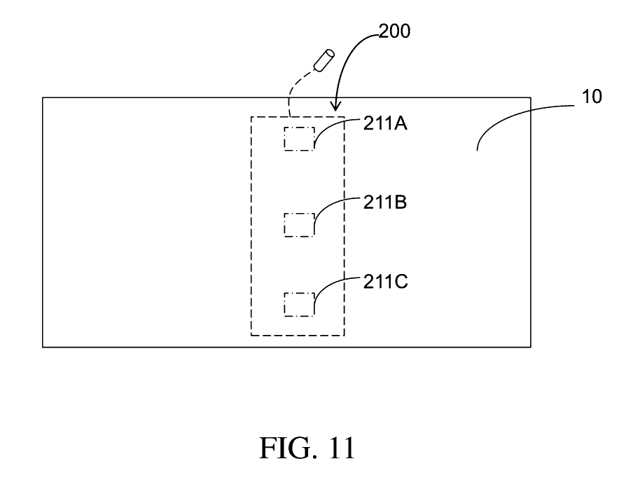

FIG. 11 illustrates a schematic diagram of an embodiment of the state determination apparatus 200, disposed on a mattress 10, wherein the blocks in dot-dashed lines indicate the first antenna modules 211A, 211B, and 211C. For example, the accuracy of the state determination can be further enhanced even if the user worn a diaper with an RFID tag as exemplified above moves oneself on the mattress 10 frequency during rest or sleeping. Further, it is noted that the embodiments in FIGS. 10 and 11 are for illustration; the invention is not limited thereto; e.g., the number and/or the positions and/or the types of the first antenna modules may be changed.

In addition, in another embodiment, a state determination system for a care-receiver is provided, comprising: an RFID tag and a state determination apparatus for a care-receiver. The state determination apparatus for a care-receiver, as exemplified in any of the above embodiments, can be disposed on a back of a cushion to perform state determination with respect to a care-receiver. In an embodiment, the RFID tag is disposed on an outer side of an absorbent article, and the state of the care-receiver is, for example, a wetness state of the absorbent article of the care-receiver, as exemplified in any of the above embodiments such as that for FIG. 3. The absorbent article is a diaper or urine pad. In another embodiment, the RFID tag is worn on the care-receiver or attached to clothes of the care-receiver, and the state of the care-receiver is a fall warning state of the care-receiver; and this embodiment can be implemented by way of the above embodiments.

As disclosed above, the state determination apparatus for a care-receiver according to the invention, is disposed on a back of a cushion (such as a mattress, seat cushion, back support cushion) to perform state determination with respect to a care-receiver, thus leading to higher accuracy of the state determination with respect to the care-receiver. In addition, as one embodiment above, the state determination apparatus for a care-receiver can be further utilized for determining whether the user leaves the position of the cushion (e.g., it may indicate that the user has left the bed or has fallen), thus providing additional functionality for caring. As such, the invention can greatly reduce the burden of work on the caregivers.

In addition, the state determination apparatus for a care-receiver according to the invention, which is disposed under a cushion, does not require additional space for hardware arrangement on the room, thereby saving hardware space and cost. The efficiency of configuring the state determination apparatus for a care-receiver in hospitals, healthcare centers, or homes can be enhanced.

In addition, the users of diapers will not feel uncomfortable since the state determination apparatus for a care-receiver is disposed on a back of a cushion (such as a mattress), not in contact with the body of the user. Since there is no noticeable special detection device equipped in the room, it will not bring additional sense of shame of being detected on the user. In addition, in one of the above embodiments, the state determination apparatus for a care-receiver informs the caregiver of the care-receiver's state by way of a wireless network linked to the computing device, so the matter making the user feeling shame, such as buzzing or flashing on the spot, will not happen.

In addition, the invention provides a state determination system for a care-receiver, wherein the RFID tag is utilized for being disposed on an outer side of a diaper, and the state determination apparatus for a care-receiver is utilized for being disposed on a back of a cushion, so as to perform state determination of the user on a front side of the cushion. Therefore, there is no need to employ or design a complicated and expensive diaper and/or bed (or chair, wheelchair) with wetness detection. The efficiency of configuring the state determination apparatus for a care-receiver in hospitals, healthcare centers, or homes can be greatly enhanced.

It will be apparent to those skilled in the art that various modifications and variations can be made to the present disclosure without departing from the spirit and scope of the disclosure. Thus it is intended that the present disclosure cover the modifications and variations of this disclosure provided they come within the scope of the appended claims and their equivalents.

What is claimed is:

1. A state determination apparatus for a care-receiver, comprising:
a communication unit, comprising:
a first antenna module; and
a first wireless communication module, coupled to the first antenna module; and
a protective unit, which at least covers the communication unit, for accommodating the communication unit and for buffering external pressure on the communication unit so as to protect the communication unit;
wherein the state determination apparatus is configured for being disposed on a back of a cushion;
the first wireless communication module sends a detection signal through at least the first antenna module so as to communicate with a radio frequency identity (RFID) tag used by the care-receiver;
after the detection signal is sent, the communication unit determines whether a response signal from the RFID tag is received or information of the response signal is read so as to determine a state of the care-receiver.

2. The state determination apparatus according to claim 1, wherein the RFID tag is disposed on an outer side of an absorbent article, the state of the care-receiver is a wetness state of the absorbent article of the care-receiver.

3. The state determination apparatus according to claim 2, wherein the absorbent article is a diaper or urine pad.

4. The state determination apparatus according to claim 1, wherein the RFID tag is worn on the care-receiver or attached to clothes of the care-receiver, the state of the care-receiver is a state of fall warning for the care-receiver.

5. The state determination apparatus according to claim 1, wherein the protective unit covers the communication unit; the protective unit includes a protection body and a liftable part on a region, in the protective unit, corresponding to at least one portion of the communication unit; when the liftable part is lifted, the portion of the communication unit is exposed.

6. The state determination apparatus according to claim 1, wherein the protective unit comprises:
a first protective layer; and
a second protective layer; wherein
the communication unit is disposed between the first protective layer and the second protective layer; the first protective layer and the second protective layer are flexible; the first protective layer has a hardness greater than that of the second protective layer.

7. The state determination apparatus according to claim 6, wherein the protective unit covers the communication unit; the protective unit has a liftable part on a region, in the first protective layer or the second protective layer of the protective unit, corresponding to at least one portion of the communication unit; when the liftable part is lifted, the portion of the communication unit is exposed.

8. The state determination apparatus according to claim 1, further comprising:
an input part, coupled to the communication unit, having at least one of a power input, a data interface, and an input key.

9. The state determination apparatus according to claim 1, further comprising:
a protective cover, wherein the protective unit and the communication unit are wrapped in the protective cover.

10. The state determination apparatus according to claim 1, wherein the communication unit further comprises:
a second antenna module; and
a second wireless communication module, electrically coupled to the first wireless communication module and the second antenna module; wherein
the first wireless communication module utilizes the second antenna module and the second wireless communication module so as to communicate with a computing device in a wireless network, and sends the state of the care-receiver to the computing device.

11. The state determination apparatus according to claim 10, wherein the communication unit determines the state of the care-receiver as an abnormal state or a state of getting wet when the response signal is not received; the communication unit determines the state of the care-receiver as a normal state or a state of getting dry when the response signal is received.

12. The state determination apparatus according to claim 10, wherein the first wireless communication module sends a detection signal through at least the first antenna module for at least one or a plurality of times so as to communicate with the RFID tag; and the communication unit determines the state of the care-receiver based on a received signal strength indicator (RSSI) in the response signal.

13. The state determination apparatus according to claim 12, wherein the state of the care-receiver is a wetness state of an article on the care-receiver; the communication unit determines the wetness state as a state of getting wet when a value based on the RSSI is within a setting range; the value based the RSSI indicates a corresponding amount of moisture of the article.

14. The state determination apparatus according to claim 10, wherein the communication unit determines the state of the care-receiver as an abnormal state or a state of getting wet when the information including an identification code in the response signal is not received; the communication unit determines the state of the care-receiver as a normal state or a state of getting dry when the information including the identification code in the response signal is received.

15. The state determination apparatus according to claim 10, wherein the communication unit comprises a plurality of the first antenna module:
a switch device, coupled between the first antenna modules and the first wireless communication module;
the first wireless communication module sends the detection signal through the first antenna modules for a plurality of times so as to communicate with the RFID tag;
after the detection is sent, the communication unit determines the state of the care-receiver based on the response signal accordingly.

16. A state determination system for a care-receiver, comprising:
a radio frequency identity (RFID) tag for being used by a care-receiver; and
a state determination apparatus, comprising:
a communication unit, including:
at least one first antenna module; and
a first wireless communication module, coupled to the at least one first antenna module; and
a protective unit, which at least covers the communication unit, for accommodating the communication unit and for buffering external pressure on the communication unit so as to protect the communication unit;
wherein the state determination apparatus is configured for being disposed on a back of a cushion;
the first wireless communication module sends a detection signal through at least the first antenna module so as to communicate with a radio frequency identity (RFID) tag;
after the detection signal is sent, the communication unit determines whether a response signal from the RFID tag is received or information of the response signal is read so as to determine a state of the care-receiver.

17. The state determination system according to claim 16, wherein the communication unit further comprises:
a second antenna module; and
a second wireless communication module, electrically coupled to the first wireless communication module and the second antenna module; wherein
the first wireless communication module utilizes the second antenna module and the second wireless communication module to communicate with a computing device in a wireless network, and sends the state of the care-receiver to the computing device.

18. The state determination system according to claim 16, wherein the first wireless communication module sends a detection signal through at least the first antenna module for at least one or a plurality of times so as to communicate with the RFID tag; and the communication unit determines the state of the care-receiver based on a received signal strength indicator (RSSI) in the response signal.

19. The state determination system according to claim 18, wherein the state of the care-receiver is a wetness state of an article on the care-receiver; the communication unit determines the wetness state as a state of getting wet when a value based on the RSSI is within a setting range; the value based the RSSI indicates a corresponding amount of moisture of the article.

20. The state determination system according to claim 16, wherein the cushion is one of a mattress, a wheelchair cushion, a back support cushion, a seat cushion, a lumbar cushion, and an air cushion.
